(19) United States
(12) Patent Application Publication
(10) Pub. No.: US 2025/0059489 A1
(43) Pub. Date: Feb. 20, 2025

(54) ARRANGEMENT FOR CLASSIFYING PLANT EMBRYOS

(71) Applicants: KAAKKOIS-SUOMEN AMMATTIKORKEAKOULU OY, Mikkeli (FI); LUONNONVARAKESKUS, Helsinki (FI)

(72) Inventors: Elmar BERNHARDT, Mikkeli (FI); Elina Katarina HAVIA, Mikkeli (FI); Tero-Markus JANKKO, Mikkeli (FI); Hannu LEINONEN, Mikkeli (FI); Henri MONTONEN, Mikkeli (FI); Mikko TIKKINEN, Helsinki (FI); Tuija ARONEN, Helsinki (FI); Sakari VÄLIMÄKI, Helsinki (FI); Saila VARIS, Helsinki (FI)

(21) Appl. No.: 18/712,574

(22) PCT Filed: Nov. 24, 2022
(86) PCT No.: PCT/FI2022/050786

§ 371 Date: May 22, 2024

(30) Foreign Application Priority Data
Nov. 25, 2021 (FI) 20216202

(51) Int. Cl. C12M 1/34; C12M 1/26; C12M 1/36

(52) U.S. Cl. CPC C12M 41/36; C12M 33/08; C12M 41/48

(57) ABSTRACT

An arrangement (100) for classifying plant embryos comprising a vibrating base (102) configured to receive. relocate and/or detach, by vibrating. one or more plant embryos (104) without liquid, at least one sensor (108) configured to at least detect a single plant embryo (104) on the vibrating base (102), a robot (106) having a gripper (110) configured to pick the detected single plant embryo (104) from the vibrating base (102), at least one camera (112) configured to image the single plant embryo (104) in the gripper (110) from a plurality of directions and a processing unit configured to determine, based on the images. a quality of the single plant embryo (104), and wherein the processing unit is further configured to provide a control signal to the robot (106) to move, based on the quality of the embryo (104), the single plant embryo (104) to a first or a second group.

### FIELD

The invention relates to a field of arrangements for plant embryos, especially arrangements for classifying the plant embryos.

### BACKGROUND

There are a plenty of commercial arrangements available on the market for processing of the plant embryos. Still, the known solutions have many drawbacks. The drawbacks may relate to contamination of the plant embryos during the processing, for example. The contamination may be fatal to the plant embryos.

Hence, there is a need for a solution to alleviate the drawbacks in the known arrangements for processing the plant embryos.

### BRIEF DESCRIPTION

The present invention is defined by the subject matter of the independent claims.

Embodiments are defined in the dependent claims.

The embodiments and features, if any, described in this specification that do not fall under the scope of the independent claim are to be interpreted as examples useful for understanding various embodiments of the invention.

### LIST OF DRAWINGS

Example embodiments of the present invention are described below, by way of example only, with reference to the accompanying drawings, in which

FIGS. 1, 3 and 4 illustrate an arrangement for classifying plant embryos according to embodiments of the invention;

FIGS. 2A, 2B and 2C illustrate arrangement for classifying the plant embryos according to other embodiments of the invention; and

FIGS. 5 and 6 illustrate flow diagrams according to embodiments of the invention.

### DESCRIPTION OF EMBODIMENTS

The following embodiments are only examples. Although the specification may refer to “an” embodiment in several locations, this does not necessarily mean that each such reference is to the same embodiment(s), or that the feature only applies to a single embodiment. Single features of different embodiments may also be combined to provide other embodiments. Furthermore, words “comprising” and “including” should be understood as not limiting the described embodiments to consist of only those features that have been mentioned and such embodiments may contain also features/structures that have not been specifically mentioned. All combinations of the embodiments are considered possible if their combination does not lead to structural or logical contradiction.

Plant embryos are very sensitive and set tight requirements for arrangements used for processing them. For example, contamination of the plant embryos shall be prevented during the processing since it may be fatal to the plant embryos. The processing may refer to a quality-based classification of the plant embryos, for example. The known solutions for classifying and handling of the plant embryos have many drawbacks and therefore more sophisticated arrangement for classifying the plant embryos is desired.

According to an aspect, there is provided an arrangement for classifying plant embryos, comprising a vibrating base configured to receive, relocate and/or detach, by vibrating, one or more plant embryos without liquid, at least one sensor configured detect a single plant embryo on the vibrating base, a robot having a gripper configured to pick the detected single plant embryo from the vibrating base, at least one camera configured to image the single plant embryo in the gripper from a plurality of directions and a processing unit configured to determine, based on the images, a quality of the single plant embryo, and wherein the processing unit is further configured to provide a control signal to the robot to move, based on the quality of the embryo, the single plant embryo to a first or a second group.

The term “classification” in this application refers to a process wherein the plant embryos are divided two or more groups based on the quality. The plant embryos which quality is good enough are moved to germination and the plant embryos whose quality is not good enough are removed. The removed embryos may be reclassified or eliminated, for example. The plant embryo(s) (also embryo later in this application) in this application may refer to a plurality of different kind of embryos. The embryo may refer to tree embryos, conifer embryos and/or vegetatively propagated embryos, for example. The embryo may be a Norway spruce (Picea abies L. Karst) embryo, for example.

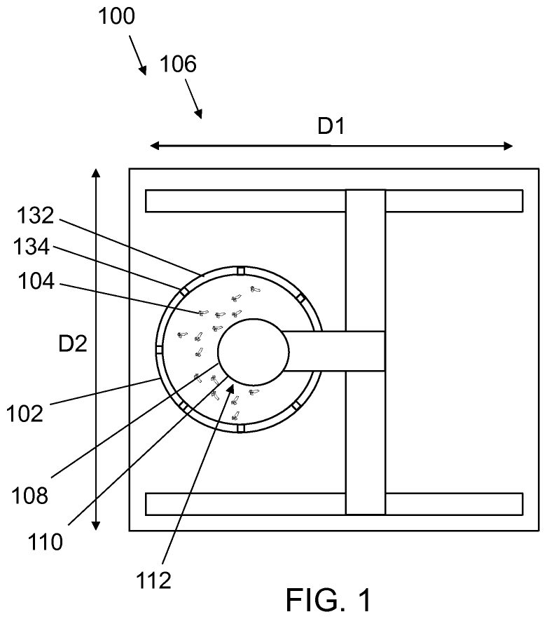

Referring to FIG. 1, in an embodiment, the arrangement 100 comprises the vibrating base 102 configured to receive, relocate, and/or detach the one or more plant embryos 104. The vibration table is coupled with a motor for providing the vibration of the base. The vibration detaches the embryos from each other and also from possible plant tissues. The vibration may also relocate the embryos on the base, in other words, the vibration may change a place of the embryo on the base. The detaching and/or relocating is performed dry, in other words, without liquid. Liquid may increase a risk of the contamination and therefore, the detaching and/or relocating process is performed without liquid. The vibrating base may comprise a wall on an outer edge of the base. The wall may extend substantially perpendicularly from a top surface of the vibration base. The wall prevents dropping of the embryos from the base during the vibration. A shape of the vibration base may a circle, for example.

The embryos are placed on the vibration base manually or automatically. For example, an operator may place the embryos on the vibration base or there may be an automated mechanism for placing the embryos. The automated mechanism may comprise a robot, for example. The robot may be the same as used in the classification or it may be a separate robot.

In an embodiment, the vibration base comprises a ceramic and/or Teflon (polytetrafluoroethylene, PTFE) coating. The coating enables easier removal of the embryos from the base since the embryos can easily get stuck to the base. In an embodiment, a colour of the vibration base is dark. Dark colour may refer to black, for example. In addition, the colour of the vibration base shall be uniform throughout the surface of the base to which the embryos are placed. The uniform dark colour of the vibration base enables better detection of embryos on the base by the at least one sensor.

Still referring to FIG. 1, in an embodiment, the arrangement 100 comprises at least one sensor 108 configured at least detect a single plant embryo 104 on the vibrating base 102. The sensor may detect the embryo on the vibrating base and the sensor further provides information about the location of the embryos on the vibrating base. The processing unit, coupled with the at least one sensor, may provide a control signal, based on the detection of the at least one sensor, to the robot having the gripper to pick the detected single embryo from the vibrating base. The sensor may be placed such that it can detect and/or locate the plant embryos on the vibration plate. Hence, the sensor may be placed substantially above the vibration plate, for example.

In an embodiment, the sensor comprises a camera. The camera may be an industrial camera, for example. The camera may be a colour or monochrome camera. A resolution may be (at least) 20 megapixels, for example.

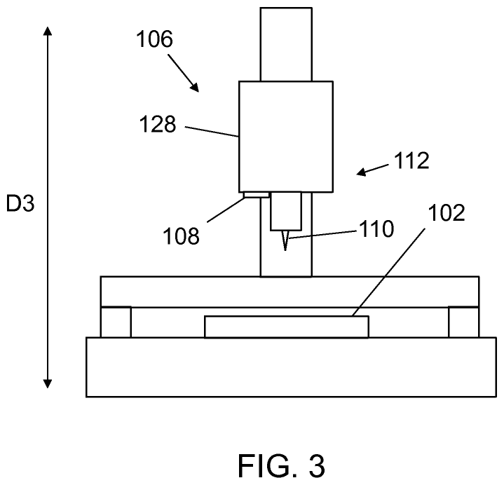

In an embodiment, illustrated in FIG. 1, the arrangement 100 comprises the robot 106 having the gripper 110 configured to pick the single plant embryo 104 from the vibrating base 102. The robot may a three-axis robot that can move in X-, Y-and Z-directions, for example. Still referring to FIG. 1, the robot may be capable of moving in D1 and D2 directions, and further in D3 direction illustrated in FIG. 3. Hence, the robot is configured to move the embryo in three different directions. The robot comprises the gripper configured to pick the single embryo from the vibration base having a plurality of the embryos. The gripper may comprise a vacuum gripper wherein the vacuum (under pressure) is used to get a grip from the single embryo. The vacuum gripper may be a needle type, for example. The vacuum gripper may have a coating to ensure proper contact with the embryo. The proper contact means that the embryo sticks to the gripper properly and stay in the gripper, and further can also be easily removed from the gripper. The coating may also reduce risks of the contamination of the embryo. The coating may be DLC-coating (Diamond Like Coating), for example. A colour of the coated gripper may be dark like black, for example.

In an embodiment, the arrangement 100 comprise at least one camera 112 configured to image the single plant embryo 104 in the gripper 110 from a plurality of directions. The single embryo means that one embryo is imaged at a time. Hence, a plurality of the embryos is imaged one by one. When the embryo is on the gripper, the imaging process is fast compared to the solution in which the embryo is set on a plane for imaging, for example. The fast imaging process reduces risks of the contamination. The at least one camera is placed such that the embryo is imaged from several angles (direction) at once. This also make the imaging process fast. A special optics may be used in the camera to get the embryo imaged from a plurality of the angles. The camera may be a 2D or 3D microscope camera, for example. It may be a colour or monochrome camera. A resolution may be (at least) 5 megapixels, for example.

In an embodiment, the arrangement comprises at least two cameras for imaging the embryo from three different directions (angles) in the gripper. One camera with the special optics may be configured to image the embryo from two different sides and one camera may be configured to imaging the embryo substantially from below, for example. Three images may be taken at the same time enabling the fast imaging process. The images from the different directions of the single embryo give comprehensive view of the quality of the embryo.

In an embodiment, the arrangement is movable. The movable may refer to the (transportable) arrangement that can be moved from one place to another. The structure of the arrangement is simple and relatively small enabling easy moving of the arrangement. The structure of the arrangement may also easily be dismantled and rebuilt again. Many of the known solutions are fixed in certain position and are not suitable for moving or relocating.

In an embodiment, the arrangement further comprises the processing unit configured to determine, based on the images taken by the cameras, the quality of the single plant embryo. The processing unit may be coupled with a memory for storing quality parameters for the embryo. Based on the images the processing unit is configured to determine the quality of the embryo. The processing unit may be configured to measure predetermined parameters from the images of the embryos and these measured parameters are compared to the quality parameters stored in the memory. The quality parameters may be determined by imaging large amount of the embryos having the high and low quality. Based on these quality parameters, limit values for the high and low quality embryos are determined.

In an embodiment, the processing unit is further configured to provide the control signal to the robot to move, based on the quality of the embryo, the single plant embryo to the first or second group after determination of the quality. The two groups comprise one group for the high quality embryos and one group for the low quality embryos. For example, the first group is for the embryos having quality that is good enough, and the second group may be for the embryos having the quality that is not good enough. Then the processing unit is configured to provide the control signal for the robot to move the imaged embryo to the first or the second group based on the determined quality level. The embryos of the first group are moved to a germination and the embryos of the second group may be moved to a trash can (are removed).

In an embodiment, the processing unit and/or the memory are an integral part of the arrangement. In another embodiment, the processing unit and the memory are external parts coupled with the arrangement. The memory may be a cloud-based database, for example. The processing unit may also be a cloud-based processor, for example. In one example, the arrangement may be coupled with a computer comprising the processing unit and the memory configured to perform all the functions mentioned in this application, for example. The processing unit and the memory are not illustrated in Figures.

The embryos of the first and/or the second group may be placed in the dish by the gripper after determination of the quality. The above mentioned arrangement may further comprise features for handling the dishes to which the embryos are set after determination of the quality. In an embodiment, the dish comprises a petri dish having a cover.

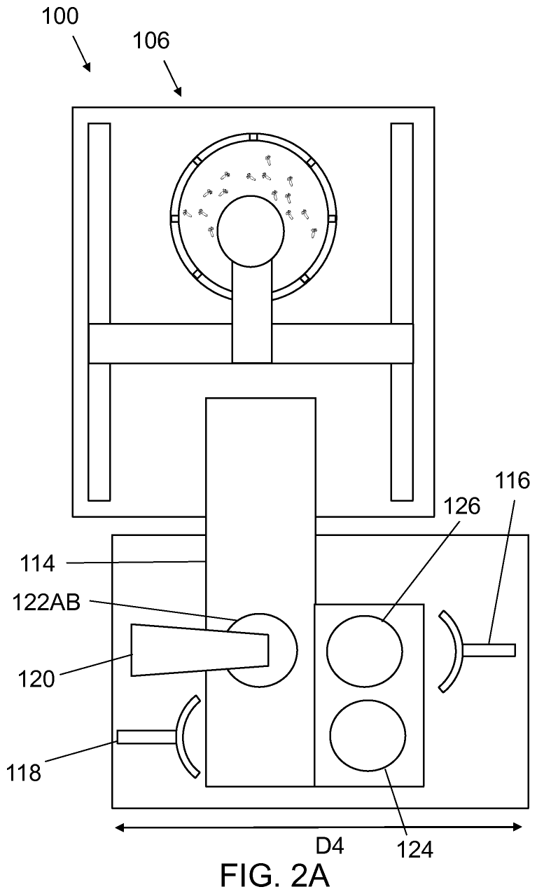

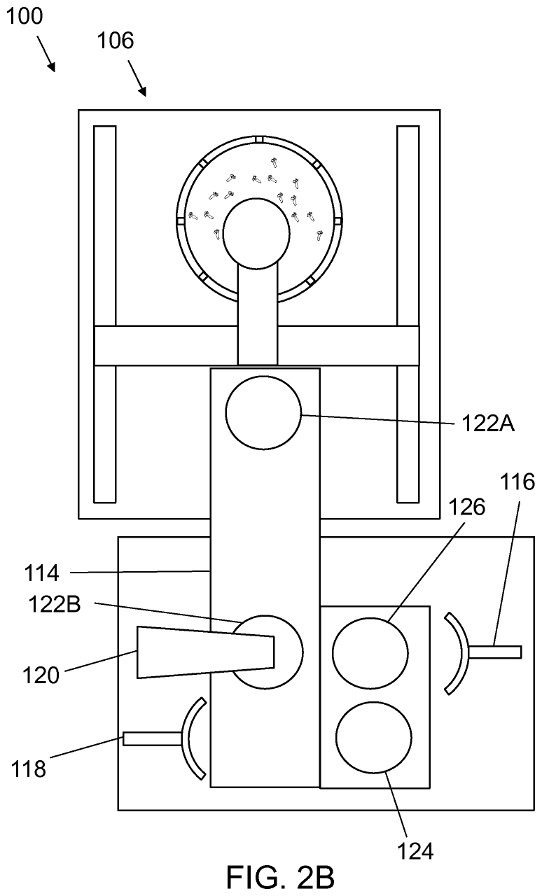

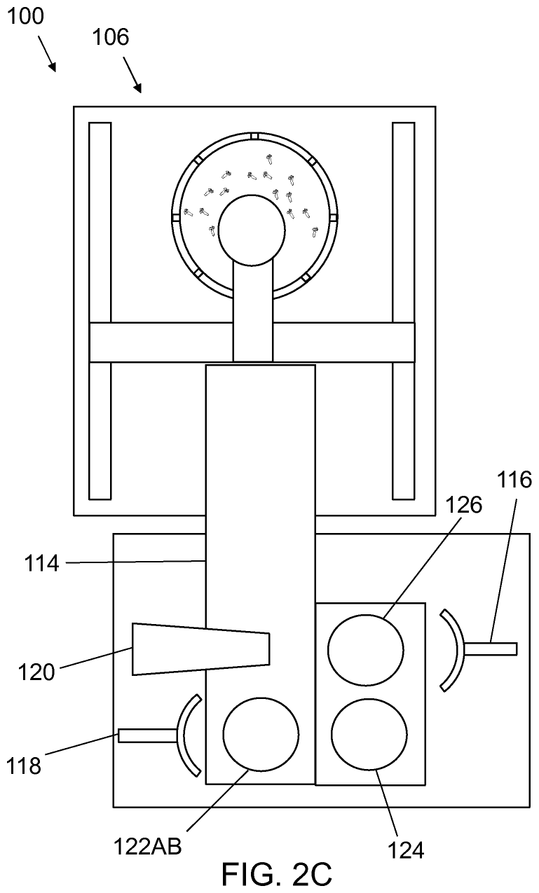

Referring to FIG. 2A, in an embodiment, the arrangement 100 further comprises a conveyor 114 configured to receive and move a dish 122AB, a first pusher 116 configured to push the dish 122AB to the conveyor 114 from a first stock of the dishes 126, a second pusher 118 configured to push the dish 122AB from the conveyor 114 to a second stock of the dishes 124 and a dish handler 120 configured to open and/or close a cover of the dish 120B. The dish handler is configured to open (remove the cover form the dish) and hold the cover of the dish when the dish is pushed on the conveyor by the first pusher, and the conveyor is configured to move the dish to a first location in which the robot is configured to set one or more embryos of the first group in the dish, and the conveyor is further configured to move the dish back to the dish handler for closing the dish by the cover, and the conveyor is further configured to move the dish to a second location in which the second pusher is configured to push the dish to the second stock of the dishes.

In an embodiment, the conveyor is a belt conveyor. In addition or instead, any other type of the conveyor may be used. The conveyor is configured to receive the dish from the stock of the dishes and move the dish to a plurality of locations. The arrangement may further comprise one or more sensors, coupled with the conveyor, configured to determine the location of the dish on the conveyor.

In an embodiment, the arrangement comprises the first and the second pusher configured to push the dish on the conveyor and/or from the conveyor. The first pusher is configured to push a new empty dish from the first stock of the dishes on the conveyor to which the one or more embryos are placed by the gripper. The second pusher is configured to push the dish with the one or more embryos away from the conveyor to the second stock of the dishes. The stock may be a pile of the dishes, for example. The first pusher may be configured to push the lowest dish of the first pile onto the conveyor and the second pusher is configured to push the dish to bottom of the second pile, for example. The pusher may comprise a cylinder coupled with a pushing head. The pushing head may have a shape of arc, for example. The dish may be a circular (round) and the arc shaped pushing head may follow the shape of the circular dish such that the dish fits into the pusher. The first and the second pusher are configured to move in direction D4 illustrated in FIG. 2A. The first and the second pusher may have substantially a similar structure.

In an embodiment, the arrangement comprises the dish handler configured to open and/or close the cover of the dish. The handler may be placed in vicinity of a place on the conveyor to which the first pusher pushes the dish from the first dish stock. The dish handler may have a vacuum gripper configured to remove the cover by lifting it from the dish and further hold the cover up for predetermined time. The handler is further configured to set the cover back to the dish.

The conveyor 114 is configured to receive the dish 122AB pushed on the conveyor 114 by the first pusher 116 from the first stock of the dishes 126 as illustrated in FIG. 2A. When the dish 122AB is on the conveyor 114, the dish handler 120 opens the cover of the dish 122B and holds the cover up. Then the conveyor 114 moves the dish without the cover 122A to the first location as illustrated in FIG. 2B. In the first location, which is in the operating range of the robot, the robot 106 sets the one or more embryos 104 of the first group in the dish 122A. When there are embryos 104 of the first group enough in the dish 122A, the conveyor 114 moves the dish 120A back to the dish handler 120 for closing the dish by the cover 122B. The conveyor 114 moves the closed dish 122AB to the second location as illustrated in FIG. 2C. In the second location, the second pusher 118 pushes the dish 122AB with embryos to the second stock of the dishes 124.

Referring to FIG. 3, in an embodiment, the arrangement 100 further comprises a tool module 128, coupled with the robot 106, comprising at least the gripper 110 and a plurality of the cameras 112. The tool unit may be coupled with an arm of the robot. The arm refers to a part of the robot to which the tools, like gripper, are coupled. The tool module may be a multifunctional unit in the robot comprising the tools needed in the handling and classifying the embryos like, for example. the gripper, the at least one sensor and/or the cameras. Hence, all the needed tools are integrated into one module. Then there are no needs for changing the tools by removing and/or coupling them from/with the robot during the process. The handing and classifying process is faster when all the needed tools are in one tool module coupled with the robot.

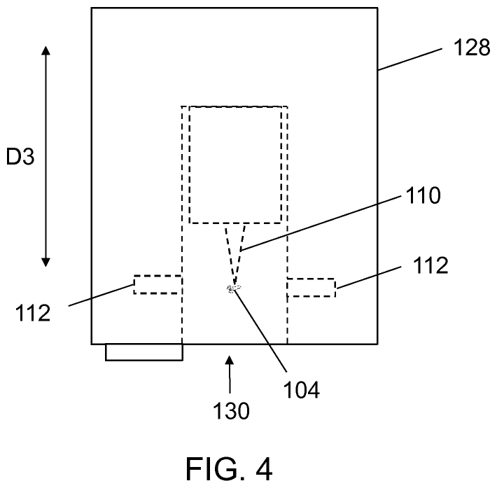

Referring to FIG. 4, in an embodiment, the tool module 128 is configured to receive the gripper 110 such that the gripper 110 moves inside the tool module 128, wherein a plurality of cameras 112 are inside the tool module 128 configured to image the single plant embryo 104 in the gripper 110 inside the tool module 128. Features that are inside the tool module are illustrated with dotted lines in FIG. 4. The tool module 128 may comprise a space 130 to which the gripper 110 may be retracted. The gripper is configured to move in the direction D3 enabling the movement of the gripper inside the tool module and out of the tool module. FIG. 4 illustrates a position of the gripper where it is inside the tool module. The gripper may be moved inside the tool module for imaging the plant embryo in the gripper. The cameras used for imaging the embryo in the gripper may be placed inside the tool module. Imaging inside the tool module improves the quality of the images and hence, also improves the determination of the quality. External interferences may be avoided when the imaging is performed inside the tool module.

In an embodiment, the at least one sensor is further configured to image the plant embryos, wherein the processing unit is further configured to identify, based on at least one image, the plant embryos on the vibrating base. In addition of the embryos, there may be the plant tissues and/or a stack of the embryos on the vibrating base, for example. The processing unit may, based on the image, identify which objects are embryos on the vibrating base. In other words, the processing unit identifies is the object on the vibrating base the embryo or not. Further, the processing unit may preliminarily determine the quality of the embryos. As described above, the at least one sensor may comprise the camera.

The camera may be used to take the image from the objects like the plant embryos and/or the plant tissues on the vibrating base. The images may be used, by the processing unit, to identify the embryos on the base before the final classification.

In an embodiment, the processing unit is configured to provide a control signal to the robot having the gripper to pick the single plant embryo from the identified objects on the vibration base. In other words, the gripper may be configured to pick the embryo(s) from the vibrating base that are identified to be the plant embryos. A technical advance of this feature is that the actual classification is enhanced since only the embryos and/or the embryos that preliminarily seem to be good enough are selected for the final imaging process. In other words, a yield of the final imaging process (classification) may be higher and the process faster.

In an embodiment, the arrangement further comprises a memory configured to store the images and/or data relating to the quality of the plant embryos. The memory may be the same as described above in this application or it may have the same features. All the classification related data, like images, measured parameters and result of the classification process, may be saved in the memory.

In an embodiment, the processing unit is configured to use the stored images and/or the data of the quality of the plant embryos in the determination of the quality of the plant embryo. The images, measured parameters and other data may be used, by the processing unit, for updating the determination process. Hence, the classification may be a self-learning process enabling the determination to come more accurate over the time based on the results of the classification and/or measured parameters and/or images.

In an embodiment, at least part of the arrangement is in an aseptic space. The arrangement or a part of it may be placed into an aseptic enclosure. The aim of the aseptic enclosure is to prevent contamination of the embryos during the handling and classifying process. The aseptic enclosure may be a laminar flow cabinet, for example. In an embodiment, at least the process steps in which the embryo is out of the dish are performed in the aseptic enclosure. In another embodiment, the whole arrangement is in the aseptic space. It is still important to realize that in the above mentioned embodiment the arrangement may be coupled with the control device (like a computer) and the control device may be out of the aseptic space nevertheless the whole arrangement is in the aseptic space. Then controlling of the arrangement may be performed out of the aseptic space.

In an embodiment, illustrated in FIG. 1, the arrangement 100 further comprises an illumination element 132 in the vibrating base 102 configured to illuminate the plant embryos 104 on the vibrating base 102 from a plurality of directions. The illumination element 132 may comprise a plurality of LED-lights 134, for example. The LED-light may be placed such that the top surface of the vibration base, where are the embryos, is illuminated from several direction. The illumination element with the top surface of the vibration base having dark colour enables accurate detection of the embryos on the surface by the at least one sensor. It also enables the high quality images of the embryos used for preliminary determination of the quality of the embryos.

Still referring to FIG. 1, in an embodiment the illumination element 132 comprises an illumination ring on an edge of the vibrating base 102. The vibrating base may be circular (round) and the illumination ring is coupled with the outer edge of the circular base such that it is circling around the outer edge of the base as illustrated in FIG. 1. There may be a plurality of the LED-lights in the illumination ring such that the LED-light are evenly distributed around the edge of the base. Then the LED-lights are configured to illuminate the top surface of the vibration base from a plurality of directions and the embryos on the surface are very efficiently illuminated. Still the illumination ring enables simple structure of the arrangement and further saves space in the arrangement.

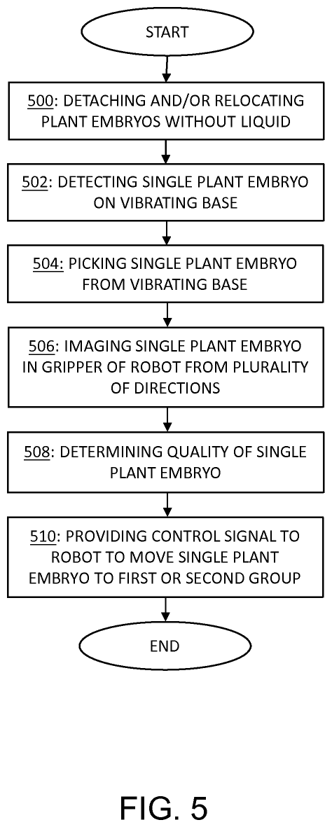

Referring to FIG. 5, according to another aspect of the invention, there is provided a method for classifying plant embryos comprising the following steps: detaching and/or relocating (step 500), by a vibrating base, one or more plant embryos without liquid, detecting (step 502), by at least one sensor, a single plant embryo on the vibrating base, picking (step 504), by a robot having a gripper, the single plant embryo from the vibrating base, imaging (step 506), by a plurality of cameras, the single plant embryo in the gripper of the robot from a plurality of directions, determining (step 508), by a processing unit, based on the images, a quality of the single plant embryo and providing (step 510), by the processing unit, a control signal to the robot to move, based on the quality of the single plant embryo, the single plant embryo to a first or a second group.

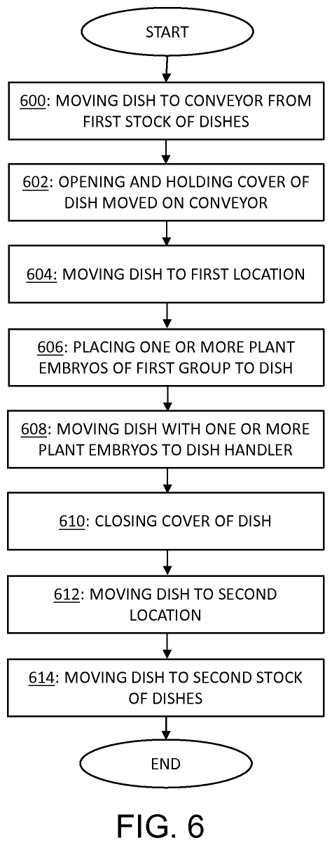

Referring to FIG. 6, in an embodiment, the method further comprises the following steps: moving (step 600), by a first pusher, a dish to a conveyor from a first stock of the dishes, opening and holding (step 602), by a dish handler, a cover of the dish moved on the conveyor, moving (step 604), by a conveyor, the dish to a first location, placing (step 606), by a gripper of the robot, one or more plant embryos of the first group to the dish, moving (step 608), by the conveyor, the dish with the one or more plant embryos to the dish handler, closing (step 610), by the dish handler, the cover of the dish, moving (step 612), by the conveyor, the dish to a second location; and moving (step 614), by a second pusher, the dish to a second stock of the dishes.

In an embodiment, the arrangement further comprises a safety device configured to stop action of the arrangement (robot) if the safety device detects an object in a predetermined safety area. For example, the safety device may stop the action (function) of the arrangement if a person puts a hand into the working area of the arrangement. The safety device may comprise one or more sensors, for example. The safety device may be a photoelectric safety switch, for example.

In an embodiment, the arrangement is further configured to mark the dishes such that each dish and/or embryo(s) on it can be traced. With the traceability, the results of the classification process of the marked dish and/or embryos may be traced later after the classification. For example, the stored data of the classification process, performed by the arrangement, may be traced with the marking of the dish, and results can be studied later. The marking may comprise a sticker and/or the marking maybe done to a surface of the dish. The marking may comprise numbers, letters and/or symbols. Instead, or in addition, it may also be a barcode and/or a QR-code, for example.

According to further aspect of the invention, there is provided a computer program product comprising instructions to cause the arrangement for classifying plant embryos to execute any of the steps of the method described above. The computer program may be in source code form, object code form, or in some intermediate form, and it may be stored in some sort of carrier, which may be any entity or device capable of carrying the program. Such carriers include transitory and/or non-transitory computer media, e.g. a record medium, computer memory, read-only memory, electrical carrier signal, telecommunications signal, and software distribution package. Depending on the processing power needed, the computer program may be executed in a single electronic digital processing unit or it may be distributed amongst a number of processing units.

The next example describes how the arrangement may be applied in the classification of the embryos. A plurality of the plant embryos are placed, manually or automatically, on the vibrating base. The vibration detaches the embryos from each other and also from the plant tissues. The at least one sensor, that may be camera, is configured to detect and locate the embryos on the vibration base and further also image the embryos on the surface of the vibration base. The data received from the at least one sensor is used, by the processing unit, to detect and locate the embryos on the surface and guiding the gripper of the robot to pick the single embryo. Data may be further used, by the processing unit, to preliminarily determine the quality of the embryos such that only the embryos which quality level is high enough are picked up from the surface. The embryos are picked up one by one for the classification process.

The robot may comprise the tool module comprising the at least one sensor, the gripper and the cameras, for example. The tool module may be further configured to receive inside the gripper with the single embryo for imaging. Inside of the tool module is a stable environment for imaging. A plurality of cameras are placed inside the tool module configured to image the single embryo from a plurality of directions at once. The imaging process, performed with a plurality of cameras inside the tool module, is fast and stable.

The processing unit is configured to determine the quality of the embryo based on the images. The processing unit may measure predetermined parameters from the images of the embryo and compare the measured parameters to the quality parameters stored in the memory. The embryos may be divided in the two groups based on the quality. The processing unit provides the control signal to move the imaged embryos to the first or second group. The embryos of the first group may be transferred to the germination and embryos of the second group may be removed. The embryos of the first group may be put into the dish like the petri dish having the cover.

The arrangement may further comprise features for providing the dish to the robot. There may be the conveyor configured to move dish to the operating range of the robot and also move dish away from the operating range when the embryo is placed into the dish. The arrangement may further comprise the first pile of the dishes from which the first pusher is configured to push the dish on the conveyor. When the dish is on the conveyor, the dish handler lifts the cover of the dish and holds it up. Then the dish without the cover is moved by the conveyor to the first location that is inside the operating range of the robot and the single embryo is set into the dish by the gripper. When the embryo or embryos are in dish, the conveyor moves the dish with the embryos back to the dish handler that puts the cover back to the dish (closes the dish). The closed dish is then moved to the second location in which the second pusher pushes the dish away from the conveyor to the second pile of the dishes.

The arrangement for classifying the plant embryos according to the invention provides very effective automated solution to alleviate the drawbacks in the known solutions. Time, when the embryo is out of the dish, is minimized that reduces risks of the contamination during the classification. A high automation rate enables fast handling of the embryo in the classification process. In addition, the classifying process is performed without liquid which also reduces the contamination risk. The liquid-free classification process according to the invention provides the solution in which the arrangement is easy to keep clean, in other words, a genetic fidelity can easily be achieved, and a risk of a genetic cross contamination is minimized.

The liquid-free process enables also scaling of the classification process to a smaller scale. The smaller size of the arrangement enables moving of the arrangement which conventionally is problematic in the known solutions. Further, a plurality of the arrangement may be coupled together to form a larger scale arrangement for classifying the embryos.

As used in this application, the term “processing unit” may refer to “circuitry” referring to all of the following: (a) hardware-only circuit implementations such as implementations in only analog and/or digital circuitry; (b) combinations of circuits and software and/or firmware, such as (as applicable): (i) a combination of processor(s) or processor cores; or (ii) portions of processor(s)/software including digital signal processor(s), software, and at least one memory that work together to cause an apparatus to perform specific functions; and (c) circuits, such as a microprocessor(s) or a portion of a microprocessor(s), that require software or firmware for operation, even if the software or firmware is not physically present.

It will be obvious to a person skilled in the art that, as technology advances, the inventive concept can be implemented in various ways. The invention and its embodiments are not limited to the example embodiments described above but may vary within the scope of the claims.

1-15. (canceled)

16. An arrangement for classifying plant embryos, comprising:
a vibrating base configured to receive, relocate, and/or detach, by vibrating, one or more plant embryos without liquid;
at least one sensor configured at least to detect a single plant embryo on the vibrating base;
a robot having a gripper configured to pick the detected single plant embryo from the vibrating base;
at least one camera configured to image the single plant embryo in the gripper from a plurality of directions; and
a processing unit configured to determine, based on the images, a quality of the single plant embryo, and wherein the processing unit is further configured to provide a control signal to the robot to move, based on the quality of the embryo, the single plant embryo to a first or a second group.

17. The arrangement of claim 16, wherein the arrangement further comprises:
a conveyor configured to receive and move a dish;
a first pusher configured to push the dish to the conveyor from a first stock of the dishes;
a second pusher configured to push the dish from the conveyor to a second stock of the dishes; and
a dish handler configured to open and/or close a cover of the dish wherein the dish handler is configured to open and hold the cover of the dish when the dish is pushed to the conveyor by the first pusher, and
the conveyor is configured to move the dish without the cover to a first location in which the robot is configured to set one or more embryos of the first group in the dish, and the conveyor is further configured to move the dish back to the dish handler for closing the dish by the cover, and
the conveyor is further configured to move the closed dish to a second location in which the second pusher is configured to push the dish to the second stock of the dishes.

18. The arrangement of claim 16, wherein the arrangement further comprises a tool module, coupled with the robot, comprising at least the gripper and a plurality of cameras.

19. The arrangement of claim 18, wherein the tool module is configured to receive the gripper inside the tool module, wherein a plurality of cameras are inside the tool module configured to image the single plant embryo in the gripper inside the tool module.

20. The arrangement of claim 16, wherein the at least one sensor is further configured to image at least the one or more plant embryos on the vibrating base, wherein the processing unit is further configured to identify, based on at least one image, the plant embryos on the vibrating base and further configured to provide a control signal to the robot having the gripper to pick the single plant embryo from the identified plant embryos.

21. The arrangement of claim 16, wherein the arrangement is movable.

22. The arrangement of claim 16, wherein the arrangement further comprises a memory configured to store the images and/or data relating to the quality of the plant embryos.

23. The arrangement of claim 22, wherein the processing unit is configured to use the stored images and/or the data of the quality of the plant embryos in the determination of the quality of the plant embryo.

24. The arrangement of claim 16, wherein at least part of the arrangement is in an aseptic space.

25. The arrangement claim 16, wherein the arrangement further comprises an illumination element in the vibrating base configured to illuminate the plant embryos on the vibrating base from a plurality of directions.

26. The arrangement of claim 25, wherein the illumination element comprises an illumination ring on an edge of the vibrating base.

27. The arrangement of claim 16, wherein arrangement comprises at least two cameras configured to image the plant embryo from three different directions.

28. A method for classifying plant embryos comprising:
detaching and/or relocating, by a vibrating base, one or more plant embryos without liquid;
detecting, by at least one sensor, a single plant embryo on the vibrating base;
picking, by a robot having a gripper, the single plant embryo from the vibrating base;
imaging, by at least one camera, the single plant embryo in the gripper of the robot from a plurality of directions;
determining, by a processing unit, based on the images, a quality of the single plant embryo; and
providing, by the processing unit, a control signal to the robot to move, based on the quality of the single plant embryo, the single plant embryo to a first or a second group.

29. The method of claim 28 further comprises:
moving, by a first pusher, a dish to a conveyor from a first stock of the dishes;
opening and holding, by a dish handler, a cover of the dish moved to the conveyor;
moving, by the conveyor, the dish to a first location;
placing, by a gripper of the robot, one or more plant embryos of the first group to the dish;
moving, by the conveyor, the dish with the one or more plant embryos to the dish handler;
closing, by the dish handler, the cover of the dish;
moving, by the conveyor, the dish to a second location; and
moving, by a second pusher, the dish to a second stock of the dishes.

30. A computer program product comprising instructions to cause an arrangement for classifying plant embryos to execute steps of method of claim 28.
